(19) United States
(12) Patent Application Publication
(10) Pub. No.: US 2025/0152062 A1
(43) Pub. Date: May 15, 2025

(54) METHOD AND A SYSTEM FOR MEASURING INSTANTANEOUS INFLOW RATE OF URINE

(71) Applicant: Bright Uro, Inc., Irvine, CA (US)

(72) Inventor: Gil Hidas, Victoria (AU)

(21) Appl. No.: 19/026,016

(22) Filed: Jan. 16, 2025

Related U.S. Application Data
(63) Continuation of application No. 17/606,653, filed on Oct. 26, 2021, now Pat. No. 12,232,873.
(60) Provisional application No. 62/840,195, filed on Apr. 29, 2019.

(51) Int. Cl. A61B 5/20; A61B 5/00; G01F 23/24; G01F 23/26

(52) U.S. Cl. CPC A61B 5/208; A61B 5/6887; G01F 23/241; G01F 23/261; A61B 2562/0214; A61B 2562/0219; A61B 2562/164

(57) ABSTRACT

The present invention provides a system for measuring instantaneous inflow rate of liquid, comprising: a receptacle, comprising an opening at a top side, a nozzle at a bottom and/or multiple holes on the side section of said receptacle configured to receive the inflow of liquid from said top opening, and simultaneously release said liquid through said nozzles; a sensing device, configured to sense the level of liquid within the receptacle and produce signals indicative of the level of liquid in the receptacle; and a processor, configured to receive said signals, calculate the level of liquid in the receptacle according to said signals; and analyze said calculations, to obtain the instantaneous inflow rate of liquid.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### FIELD

The presented invention generally relates to the field of medical devices, and more specifically to methods of urine flow measurement and analysis.

### DISCUSSION OF RELATED ART

Uroflowmetry is the measurement of urine flow. For humans, urine voiding events have a flow rate in the range of 5 to 50 ml/second. Typical flow rates for healthy adults are in the range of 15 to 25 ml/sec, depending on the age and gender of the patient. Abnormal urine flow may have patterns of tower, plateau, interrupted or staccato and/or may be less than 10 ml/sec. Such deviations from normal urine flow may be indicative of various medical conditions, such as urinary tract obstruction, overactive urine bladder or underactive urine bladder.

In order for urine flow meters to be effective as physician support systems, they must be able to relate to the full dynamic range of expected urinary flow. They must also be able to detect the commencement of low flow rate periods and individual droplets, to indicate an interrupted or restricted flow.

Urine flow meters should mitigate the inertia effects of urine hitting the device at various angles, or falling from different heights, and prevent high velocity urine from flowing directly onto the measurement device and disrupting flow measurement.

### SUMMARY

The present invention discloses a system for measuring instantaneous inflow rate of liquid, such as water or urine, comprising:
a receptacle, comprising an opening at a top side and a nozzle on a lateral side, configured to receive the inflow of liquid from said top opening, and simultaneously release said liquid through said nozzle;
a sensing device, configured to sense the level of liquid within the receptacle and produce signals indicative of the level of liquid in the receptacle; and
a processor, configured to receive said signals, calculate the level of liquid in the receptacle according to said signals; and analyze said calculations, to obtain the instantaneous inflow rate of liquid.

According to some embodiments of the present invention the sensing device comprises at least one electrode, placed along an inner side of a lateral wall of the receptacle, such that an increasingly longer portion of the at least one electrode is covered with liquid as liquid level (H) rises, wherein the sensing device is configured to sense specific physical qualities including at least one of resistance, or capacitance of the liquid as a function of the portion of the electrode that is covered by liquid, wherein the sensing device produces signals that are indicative to the sensed physical qualities, and propagate the signals to the processor.

According to some embodiments of the present invention the sensing device is a resistive sensor which comprises three electrodes, wherein the sensing device is configured to sense the resistance of liquid covering said three electrodes and produce signals that are indicative of the liquid level H.

According to some embodiments of the present invention the sensing device comprising three electrodes which create an electric circuit:
a first electrode comprises insulated and uninsulated portions, wherein the insulated part is placed along the inner side of the receptacle wall and uninsulated part on the bottom of the receptacle.
second electrode which is completely uninsulated, that runs along the inner side of the receptacle.
wherein an excitation electric voltage signal (Vin) is applied to the second electrode a third electrode completely uninsulated, running along the inner side of the receptacle serving as a common reference (GND) to the first and second electrodes, wherein the electronic circuit is configured to sense an output voltage (Vout) between the third and the second electrodes to produce signals that are indicative of Vout to the processor.

According to some embodiments of the present invention the processor is configured to calculate the inflow Qin by applying the steps of:
calculate H according to the known Vin and said signals that are indicative of Vout, via;
calculate dH/dt by differentiating H values pertaining to consequent samples of Vout; and
calculate A (H) according to H, and the known geometry of the receptacle;

According to some embodiments of the present invention the level sensing operation is based on measuring the relative conductance between two electrodes and a series of multiple receiving pads electrodes that are spaced apart vertically.

According to some embodiments of the present invention the value of Qout as a function of H is empirically measured during a calibration process, to produce a lookup table, associating a given liquid level H with a momentary value of liquid outflow Qout.

According to some embodiments of the present invention the value of Qout as a function of H is dependent upon structural parameters of the receptacle and the outlet section, therefore computable according to parameters of the receptacle's dimensions.

According to some embodiments of the present invention the system of claim 1 wherein the outlet nozzle is between 4-7 mm, thus enhancing the linearity of Qout as a function of H throughout the entire expected flow range.

According to some embodiments of the present invention the multiple holes on the side section of said receptacle are configured, for enhancing the linearity of Qout as a function of H throughout the entire flow range.

According to some embodiments of the present invention the systems further comprising an accelerometer which measures the tilt of the system to compensate for deviation liquid level measurement.

According to some embodiments of the present invention the respectable is comprised of: an inner element having a cylindrically-shaped part and a dome-shaped upper part, wherein the dome-shaped upper part absorbs the kinetic energy of the fluid flowing into open receptacle, eliminating the effects of urine disposal height and orientation, and enabling the apparatus to measure urine flow as the momentary quantity of disposed urine;

According to some embodiments of the present invention the upper dome comprises a plurality of openings along its circumferential side walls and similarly, inner element comprises a plurality of openings along its circumferential side wall, hence the dome-shaped upper part spreads the flow evenly across the inner walls of the open receptacle.

### BRIEF DESCRIPTION OF THE DRAWINGS

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

FIG. 1 presents an elevated isometric exploded view of an embodiment of the system configured to be placed over a toilet bowl or seat.

FIG. 2 presents an elevated, isometric exploded view of an embodiment of the system installed over a toilet bowl or seat.

FIG. 3A illustrates a side view of an embodiment of an open receptacle showing an outlet nozzle.

FIG. 3B illustrates a side view of an embodiment of an open receptacle showing an outlet nozzle.

FIG. 4A illustrates the inner structure of an embodiment of the open receptable.

FIG. 4B illustrates an isometric side view of an embodiment of the open receptacle.

FIG. 5A illustrates a front view of an embodiment of the open receptacle.

FIG. 5B is a magnified view of an embodiment of an outlet nozzle.

FIG. 5C illustrates a front view of an embodiment of the open receptacle with the outlet nozzle and discharge holes.

FIG. 5D is a magnified view of an embodiment of the nozzle and discharge holes.

FIG. 6 presents an elevated, isometric view of an embodiment of the open receptacle.

FIGS. 7 and 8 illustrate a side view and a perspective top view, respectively, of an embodiment of the open receptacle with a sensing device.

FIG. 9 illustrates an embodiment of an electric circuit equivalent to the constellation of electrodes depicted in FIG. 8.

FIG. 10 illustrates an embodiment of the system hardware.

FIG. 11 illustrates an example accelerometer useable by the system in accordance with aspects of the present disclosure.

### DETAILED DESCRIPTION

The present invention discloses a system and a method for easy, accurate and reproducible measurement of an instantaneous inflow rate (Qin) and volume of liquid such as water or urine into a receptacle.

In the case of urine inflow measurement, the system is configured to:
Be fitted within standard toilet pans, which may require limited height and volume;
Be evacuated from urine through normal operation, which may not require manual evacuation of a receptacle (flow through); and
Measure inflow rates from 5 to 50 ml/sec, and practically unlimited volume, thus covering the entire scope of human biological variations.

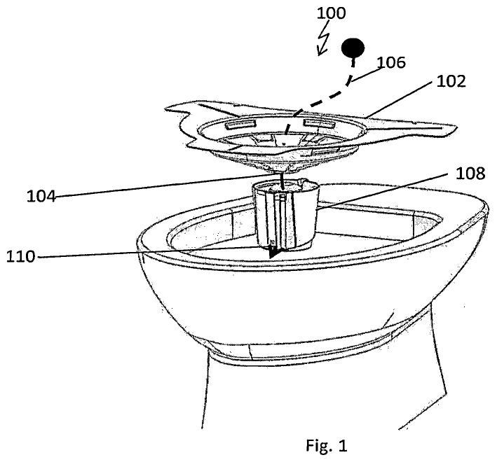

FIG. 1 presents an elevated, isometric exploded view of the system 100, as it is configured to be placed over a toilet bowl or seat, according to some embodiments of the present invention. The system 100 comprises:
a receptacle bowl 102 configured to be placed over a toilet bowl or seat, and direct fluid through a single point of exit 104;
incoming fluid 106 from the receptacle bowl 102 allowing the fluid 106 to proceed towards an open receptacle 108.
open receptacle 108 configured to receive the inflow of liquid from an opening at its top side, and simultaneously release the liquid through an outlet nozzle 110 at the bottom section of said open receptacle 108, for instance, on a lateral side towards the toilet drain;
a sensing device (not shown), configured to sense the Level of Liquid (H) within the receptacle 108 and produce signals indicative of H; and
a processor (not shown), configured to produce the calculated instantaneous inflow rate (Qin) of fluid 106 from the signals.

In accordance with some embodiments of the present invention, outlet nozzle 110 may be an outlet slit, a strainer and the like.

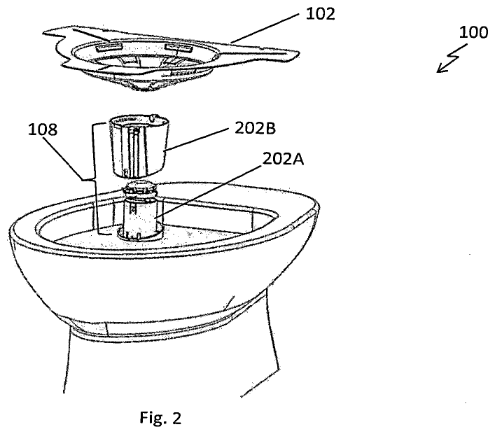

FIG. 2 presents an elevated, isometric exploded view of system 100, as it is installed over a toilet bowl or seat, according to some embodiments. Seen in the figure, open receptacle 108 comprises an inner element 202A and an outer element 202B the roles of which are described below. In these embodiments, open receptacle 108 is configured to directly attach to the receptacle bowl 102.

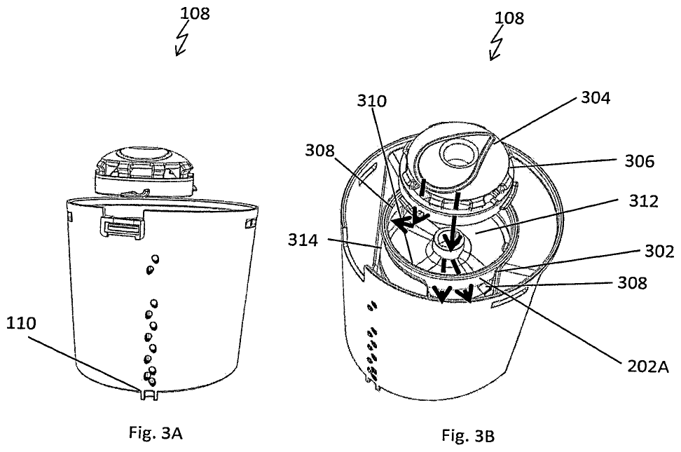

FIG. 3A illustrates a side view of open receptacle 108 showing outlet nozzle 110, and FIG. 3B illustrates a perspective view of open receptacle 108.

Seen in FIG. 3B, inner element 202A. A cylindrically shaped part 302 and a dome-shaped upper part 304. The upper part 304 has discharge holes on the lateral side, this configuration enables more consistence and linear outflow.

The dome-shaped upper part 304 serves multiple purposes:
the dome-shaped upper part 304 absorbs the kinetic energy of the fluid flowing into open receptacle 108, eliminating the effects of urine disposal height and orientation, and enabling the apparatus to measure urine flow as the momentary quantity of disposed urine.
the existence of bubbles in the inspected urine has a potentially disruptive effect on the accuracy of the fluid flow rate measurement, as bubbles acquire volume within the receptacle 108. The dome-shaped upper part 304 utilizes the fluid tension produced when the fluid contacts the dome to extract air bubbles from the fluid.
turbulence of fluid flowing into the receptacle also has a potentially disruptive effect on the accuracy of the fluid flow rate measurement. The dome-shaped upper part 304 reduces turbulence by receiving a turbulence flow and delivering a laminal flow through the dome's walls, into the open receptacle 108.

The dome-shaped upper part 304 spreads the flow evenly across the inner walls of the open receptacle 108. As seen in the figure, dome-shaped upper part 304 is not sealed but rather comprises a plurality of openings 306 along its circumferential side walls. Similarly, inner element 302 comprises a plurality of openings 308 along its circumferential side wall 310. Such openings elaborate the flow path of liquid as indicated by the arrows through the system. As the liquid hits the dome-shaped upper part 304, it flows and enters through openings 306 into the antechamber 312 underneath dome-shaped upper part 304. The liquid flows through the semi-circular openings 308 at the cylindrically shaped bottom part 302 into the internal section 314 of open receptacle 108.

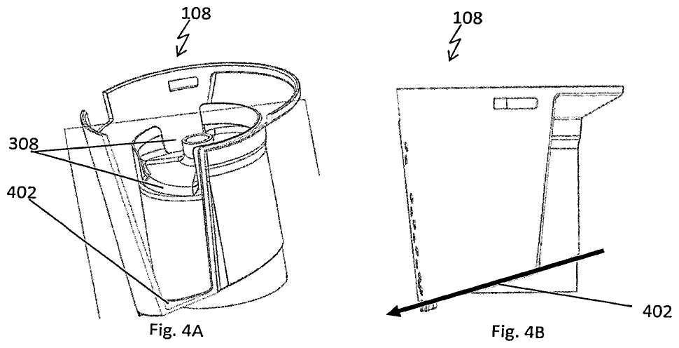

FIG. 4A illustrates the inner structure of open receptacle 108 in accordance with some embodiments of the present invention. Seen in the figure is a plurality of semi-circular openings 308 through which liquid is flowing into the internal section 314 of open receptacle 108.

In accordance with some embodiments of the present invention, open receptacle 108 is designed to enable flow rate measurements of the liquid flowing therein, based on which the volume flow and emptying time can then be calculated and extrapolated. FIG. 4B illustrates an isometric side view of open receptacle 108. Seen in the figure, open receptacle 108 is designed in a way that at the end of a flow test, it must be self-emptied, thus, as seen in the figure, open receptacle 108 comprises a sloping floor 402 to allow proper drainage.

In accordance with some embodiments of the present invention, open receptacle 108 is designed to fit within every toilet depth and therefore may be as short as 70 mm.

In accordance with some embodiments of the present invention, open receptacle 108 has separation of heights for every change in flow rate, as instantaneous flow changes (for example, distinct between 20 mlps flow rate and 19 mlps flow rate).

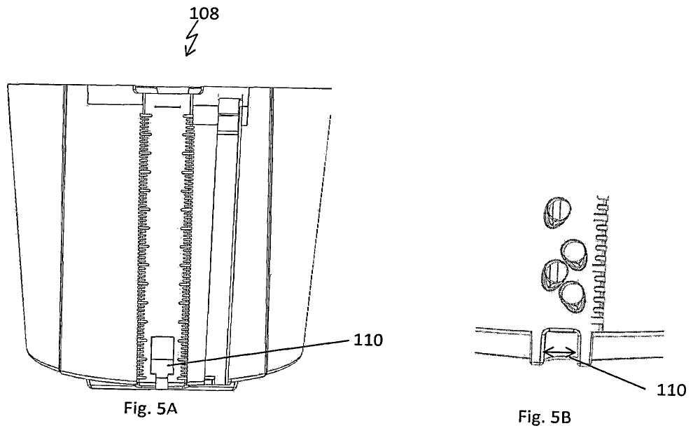

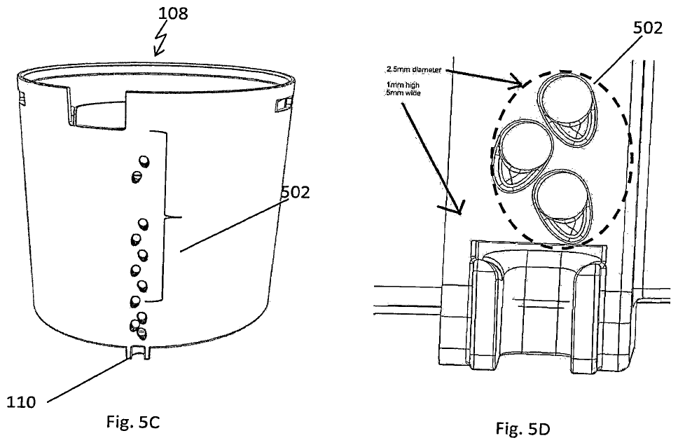

In accordance with some embodiments of the present invention as seen in FIG. 5A, open receptacle 108 is designed to have an optimal orifice outlet size or multiple orifices outlets (as seen FIG. 5C) of discharge holes to allow a linear flow rate separation as instantaneous flow rate is changing.

In accordance with some embodiments, an aim of the present invention is to have residual urine in open receptacle 108 while urine flows in and out of outlet nozzle 110. The discharge flowrate via outlet nozzle 110 is changing as the residual in the container is changing. Therefore, in accordance with some embodiments of the present invention, the size of nozzle 110 and the dimensions of open receptacle 108 are optimized so that changes in the flowrate of urine entering outlet nozzle 110 may be reflected in changes in both the residual urine height within open receptacle 108 and the discharge rate. For instance, in case the outlet nozzle 110 is not optimal, the residual urine height within open receptacle 108 is same for flow rates of 30 mlps and 33 mlps. The reason for this is that gravitational pressure presses down on the residual urine and if outlet nozzle 110 is limited, there may be a scenario where the discharge rate out of outlet nozzle 110 is greater but the residual urine height within open receptacle 108 remains the same. Such scenario may result in the inability to separate flow rates.

In accordance with some embodiments of the present invention, an optimal design of open receptacle 108, i.e., a design which enables the separation of flow rates is illustrated below in FIGS. 5C&D.

FIG. 5A illustrates a front view of open receptacle 108, and FIG. 5B is a magnified view of an outlet nozzle 110. Seen in the figure, outlet nozzle 110 may be relatively narrow to allow for measurements of relatively low flow rates. For instance, outlet nozzle 110 may be about 4-7 mm in width, and preferably 5 mm and 0.5 to 2 mm high and preferably 1 mm to allow a flow rate of about 3 mlps. Such narrow outlet section leads to relatively low flow rates which enable the sensing device to measure changes in liquid height within open receptacle 108.

FIG. 5C illustrates a front view of open receptacle 108 with outlet nozzle 110 and a series of discharge holes 502 through which liquid is draining out of open receptacle 108. Such combination of outlet nozzle 110 and multiple discharge holes 502 reflects changes in the flowrate of urine entering outlet nozzle 110 on both the residual urine height within open receptacle 108 and the discharge rate, i.e., producing a highly linear discharge rate.

In accordance with some embodiments of the present invention, the number of holes 502 as well as their size, shape, and position in open receptacle 108 may vary for optimizing the resulting discharge rate.

FIG. 5D is a magnified view of nozzle 110 and discharge holes 502 in accordance with some embodiments of the present invention.

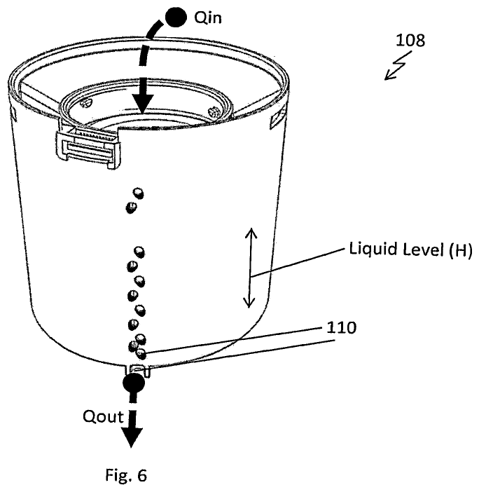

FIG. 6 presents an elevated, isometric view of open receptacle 108, according to some embodiments of the present invention.

The evacuation of liquid via outlet nozzle 110 facilitates momentary measurement of Qin, rather than deduction of Qin from differential measurement of the volume of liquid accumulated within the receptacle.

Outlet nozzle 110 is configured to evacuate the liquid from open receptacle 108 at an outflow rate Qout, which is a single-variable function of the level of liquid within the receptacle (H). Qout is therefore directly computable from the calculated H produced by the processor. The nozzle is located at the lowest gravitational point and therefore enabling consistence and measurable height to discharge ration in addition to ensuring that a full drainage is achieved.

According to some embodiments, the value of Qout as a function of H is empirically measured during a calibration process, so as to produce a lookup table, associating a given liquid level H with a momentary value of liquid outflow Qout.

According to some embodiments, the value of Qout as a function of H is dependent upon structural parameters of the receptacle and the outlet section (e.g., inclination of receptacle walls, width of the outlet section, etc.), and is therefore computable according to parameters of the receptacle's dimensions.

According to some embodiments, outlet nozzle 110 is relatively narrow, about 2 mm, thus enhancing the linearity of Qout as a function of H throughout the entire expected flow range, i.e., between 5 and 50 ml per second.

The evacuation of liquid via outlet nozzle 110 provides the following benefits to the present invention:
Liquid is not stored within the receptacle at any stage. This facilitates a reduced form factor, resulting in reduced receptacle volume and sensor size. This is particularly advantageous when the system is utilized for measuring inflow of urine, enabling the installation of the system within a toilet bowl.
It is not necessary to clear or clean the receptacle at the end of the measurement. This is also particularly advantageous when measuring inflow of urine.

According to some embodiments of the present invention, the sensing device comprises at least one electrode, placed along an inner side of a lateral wall of the receptacle, such that an increasingly longer portion of the electrode(s) is covered with liquid as H rises. The sensing devices are configured to sense specific physical qualities (e.g., resistance, capacitance) of the liquid as a function of the portion of the electrode that is covered by liquid. The sensing device produces signals that are indicative to the sensed physical qualities, and propagate the signals to the processor. The processor is configured to calculate H according to said signals, and analyze the calculations to obtain the instantaneous inflow rate of liquid (Qin) through the following formula:

$$Qin = ( \frac{dH}{dt} * A ( H ) ) - Qout \qquad [ eq . 1 ]$$

Wherein:
Qin is the instantaneous inflow rate through the opening at the receptacle's top side;
Qout is the instantaneous outflow rate from the receptacle via outlet nozzle 110 at the specified H;
dH/dt is the time-derivative of H (Liquid level in the receptacle); and
A (H) is the surface area of liquid at liquid level H (a known function of the receptacle's geometry).

The function and different embodiments of the sensing devices are further elaborated below.

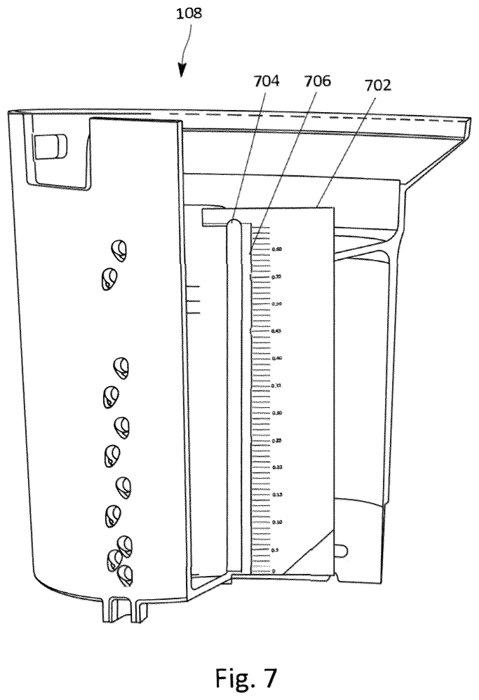

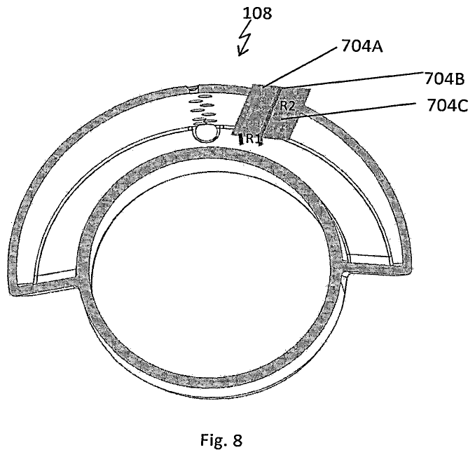

FIGS. 7 and 8 illustrate a side view and a perspective top view of the open receptacle 108 with sensing device 702, according to some embodiments of the present invention. As seen in FIG. 7, the sensing device 702 is a resistive sensor comprising electrodes 704 A-C and pads 706. The sensing device 702 is configured to sense the resistance of liquid covering said electrodes 704 A-C and pads 706 and produce signals that are indicative of the liquid level H. The signals, at each pad location, are devoid of the effect of variable resistivity between measured liquids, e.g., due to varying concentrations of Total Dissolved Solids (TDS).

As seen in FIG. 8, sensing device 702 comprising 3 electrodes, Electrode 704A, Electrode 704B, and Electrode 704C.
Electrode 704A comprises insulated and uninsulated parts (may be implemented in different configurations). The electrode is placed along the inner side of the receptacle wall (insulated) and the bottom of the receptacle (uninsulated).
Electrode 704B is completely uninsulated. An excitation electric voltage signal (Vin) is applied to the top, insulated portion of electrode 704B.
Electrode 704C is also completely uninsulated. It runs along the inner side of the receptacle 108, serving as a common reference (GND) to electrodes 704A and 704B.

The three electrodes create an electronic circuit that is configured to sense an output voltage (Vout) between electrodes 704A and 704B (R1), and between 704B and 704C (R2) produces signals that are indicative of Vout to the processor.

As soon as liquid flows into the receptacle, the resistance R1 between electrodes 704A and 704B is reduced due to conduction through the liquid at the base of the receptacle.

The sensing device is configured to sense the electric capacitance between the respective pairs of electrodes, and produce signals that are indicative of the liquid level H. The said signals are devoid of the effect of variable capacitance between different measurements that may arise, for example, from variations in the permittivity (dielectric constant) of the measured liquids or different environmental conditions in which the measurements have taken place (e.g., temperature).

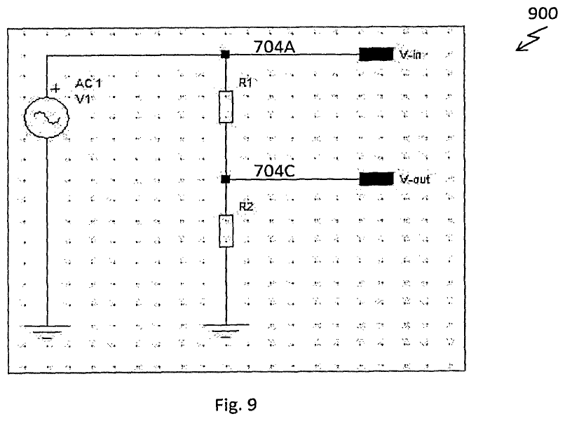

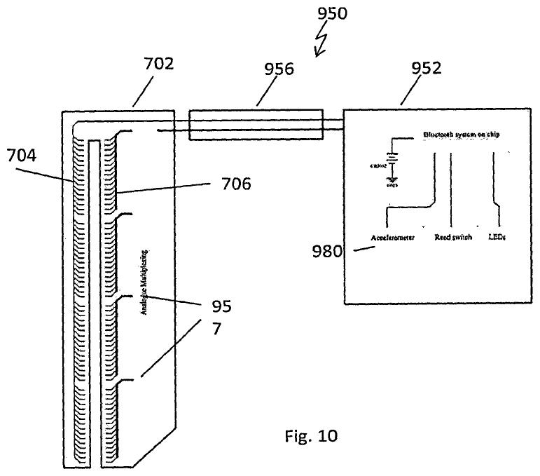

FIG. 9 illustrates an electric circuit 900 that is equivalent to the constellation of electrodes 704 A-C depicted in FIG. 8, for explaining the behavior of the resistance throughout the process of liquid level change.

An excitation electric voltage signal (Vin) is applied to electrode 704B. The electronic circuit is configured to sense an output voltage (Vout) between electrodes 704B and 704C.

As soon as liquid flows into the receptacle, the resistance R1 between electrodes 704A and 704B is reduced due to conduction through the liquid at the base of the receptacle and therefore, R1 remains constant throughout the measurement, and is not affected by the liquid level H.

As the level of liquid H increases, the resistance R2 between electrodes 704 B and C is proportionally reduced due to conduction through the liquid.

The liquid level H can therefore be expressed as:

$$H = K ( \frac{{V}_{i n}}{{V}_{o u t}} - 1 ) \qquad [ eq . 2 ]$$

wherein K is a constant which may be empirically measured during a stage of calibration. Note that the above expression is devoid of resistance or resistivity factors, and is therefore indifferent to the conductivity of the liquid.

According to some embodiments, the processor is configured to calculate the inflow Qin by applying the steps of:
calculate H according to the known Vin and said signals that are indicative of Vout, via [eq. 2];
calculate dH/dt by differentiating between H values pertaining to consequent samples of Vout;
calculate A (H) according to H, and the known geometry of the receptacle;
calculate Qout according to said lookup table or known function of Qout (H); and
obtain Qin according to above, via [eq. 1].

FIG. 10 illustrates the system hardware 950 in accordance with some embodiments of the present invention.

The system hardware 950 comprising two separate circuit boards: control board 952 and ruler board 702 joined with an FFC ribbon cable 956.

The control board 952 comprising the main processing hardware, power management, signal processing and either wired or wireless connection.

The ruler board 702 contains the sensor hardware that provides an analogue signal level back to the control board 952 to indicate liquid level.

In accordance with some embodiments of the present invention, the level sensing mechanism is based on measuring the relative conductance between an electrode (stimulus transmitter) 704 signal (e.g., 3V, 1 kHz square wave) and a series of 64 receiving ‘Pads’ electrodes 706 that are spaced 1 mm apart vertically.

The system is not required to accurately measure the absolute conductance and can compare the relative conductance between the electrode and pads in case there is liquid presence in the respectable or not. As the pads are spaced at discrete steps of 1 mm, it is possible to detect which pad is immersed in liquid and which is not.

For measuring the conductance of each pad, a series of analogue multiplexers 957 which are used to scale down the 64 individual sensors into 4 channels of 16. Each of the 4 channels has its own dedicated analogue to digital converter configured to read the signal.

### Supporting Hardware

In accordance with some embodiments of the present invention, Control Board 952 may be based on a system on chip device such as, for instance, the Nordic nRF52 system to read the pads and transmit the data via wireless connection.

In addition to that, control board 952 may also contain supporting hardware to display indicators, measure the tilt level and detect while in shipping mode.

### Accelerometer

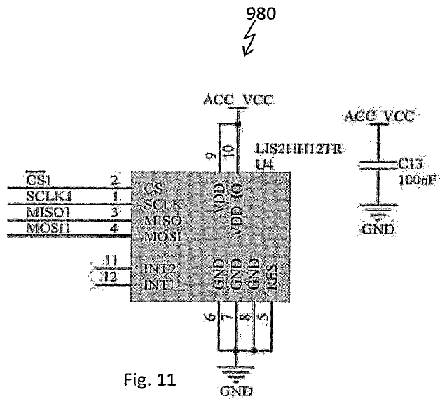

The accelerometer is configured to measure deviation of the toilet tilt in relation to the ground surface, for compensating the calculation of the liquid level. For example, an accelerometer 980, such as a 3 axis digital MEMS accelerometer 980 as seen in FIGS. 10 and 11, may be included for measuring the tilt of the system in relation to the surface. A tilt may exist due to uneven installation of toilet facility. In case of receiving a measurement from the accelerometer which indicates of a significant deviation in the tilting of the toilet, (for example, more than 2 degrees on one of the axes) compensation calculation is applied to the liquid level sensing algorithm, recalculating the levels based on the measured deviation.

The system of the present invention may include, according to certain embodiments of the invention, machine readable memory containing or otherwise storing a program of instructions which, when executed by the machine, implements some or all of the apparatus, methods, features and functionalities of the invention shown and described herein. Alternatively or in addition, the apparatus of the present invention may include, according to certain embodiments of the invention, a program as above which may be written in any conventional programming language, and optionally a machine for executing the program such as but not limited to a general purpose computer which may optionally be configured or activated in accordance with the teachings of the present invention. Any of the teachings incorporated herein may, wherever suitable, operate on signals representative of physical objects or substances.

Unless specifically stated otherwise, as apparent from the following discussions, it is appreciated that throughout the specification discussions, utilizing terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining” or the like, refer to the action and/or processes of a computer or computing system, or processor or similar electronic computing device, that manipulate and/or transform data represented as physical, such as electronic, quantities within the computing system's registers and/or mem01ies, into other data similarly represented as physical quantities within the computing system's memories, registers or other such information storage, transmission or display devices. The term “computer” should be broadly construed to cover any kind of electronic device with data processing capabilities, including, by way of non-limiting example, personal computers, servers, computing system, communication devices, processors (e.g. digital signal processor (DSP), microcontrollers, field programmable gate array (FPGA), application specific integrated circuit (ASIC), etc.) and other electronic computing devices.

The present invention may be described, merely for clarity, in terms of terminology specific to particular programming languages, operating systems, browsers, system versions, individual products, and the like. It will be appreciated that this terminology is intended to convey general principles of operation clearly and briefly, by way of example, and is not intended to limit the scope of the invention to any particular programming language, operating system, browser, system version, or individual product.

It is appreciated that software components of the present invention including programs and data may, if desired, be implemented in ROM (read only memory) form including CD-ROMs, EPROMs and EEPROMs, or may be stored in any other suitable typically non-transitory computer-readable medium such as but not limited to disks of various kinds, cards of various kinds and RAMs. Components described herein as software may, alternatively, be implemented wholly or partly in hardware, if desired, using conventional techniques. Conversely, components described herein as hardware may, alternatively, be implemented wholly or partly in software, if desired, using conventional techniques.

Included in the scope of the present invention, inter alia, are electromagnetic signals carrying computer-readable instructions for performing any or all of the steps of any of the methods shown and described herein, in any suitable order; machine-readable instructions for performing any or all of the steps of any of the methods shown and described herein, in any suitable order; program storage devices readable by machine, tangibly embodying a program of instructions executable by the machine to perform any or all of the steps of any of the methods shown and described herein, in any suitable order; a computer program product comprising a computer useable medium having computer readable program code, such as executable code, having embodied therein, and/or including computer readable program code for performing, any or all of the steps of any of the methods shown and described herein, in any suitable order; any technical effects brought about by any or all of the steps of any of the methods shown and described herein, when performed in any suitable order; any suitable apparatus or device or combination of such, programmed to perform, alone or in combination, any or all of the steps of any of the methods shown and described herein, in any suitable order; electronic devices each including a processor and a cooperating input device and/or output device and operative to perform in software any steps shown and described herein; information storage devices or physical records, such as disks or hard drives, causing a computer or other device to be configured so as to carry out any or all of the steps of any of the methods shown and described herein, in any suitable order; a program pre-stored e.g. in memory or on an information network such as the Internet, before or after being downloaded, which embodies any or all of the steps of any of the methods shown and described herein, in any suitable order, and the method of uploading or downloading such, and a system including server/s and/or client/s for using such; and hardware which performs any or all of the steps of any of the methods shown and described herein, in any suitable order, either alone or in conjunction with software. Any computer-readable or machine-readable media described herein is intended to include non-transitory computer- or machine-readable media.

Any computations or other forms of analysis described herein may be performed by a suitable computerized method. Any step described herein may be computer-implemented. The invention shown and described herein may include (a) using a computerized method to identify a solution to any of the problems or for any of the objectives described herein, the solution optionally include at least one of a decision, an action, a product, a service or any other information described herein that impacts, in a positive manner, a problem or objectives described herein; and (b) outputting the solution.

The scope of the present invention is not limited to structures and functions specifically described herein and is also intended to include devices which have the capacity to yield a structure, or perform a function, described herein, such that even though users of the device may not use the capacity, they are, if they so desire, able to modify the device to obtain the structure or function.

Features of the present invention which are described in the context of separate embodiments may also be provided in combination in a single embodiment.

For example, a system embodiment is intended to include a corresponding process embodiment. Also, each system embodiment is intended to include a server-centered “view” or client centered “view”, or “view” from any other node of the system, of the entire functionality of the system, computer-readable medium, apparatus, including only those functionalities performed at that server or client or node.

1.-13. (canceled)

14. A system for determining urine inflow rate, the system comprising:
a bowl configured for placement on a portion of a toilet, the bowl comprising a bowl outlet and an interior bowl surface configured to guide urine that flows into the bowl toward the bowl outlet;
a housing configured to receive urine from the bowl, the housing comprising a top end configured to be attached to the bowl proximate the bowl outlet, a bottom end opposite the top end, an outer wall, an inner wall arranged within and spaced from the outer wall, a first fluid chamber at least partially defined by the inner wall and proximate to the top end of the housing, a plurality of openings arranged along a perimeter of the inner wall, a bottom wall extending between the outer and inner walls, a second fluid chamber defined at least partially between the outer and inner walls and above the bottom wall, and a housing outlet arranged proximate an intersection of the outer wall and the bottom wall;
a dome-shaped part arranged above the first fluid chamber and proximate the bowl outlet when the housing is attached to the bowl, the dome-shaped part configured to provide a contact surface for urine, the system configured such that urine that contacts said interior bowl surface or the dome-shaped part is guided towards the bowl outlet, into the first fluid chamber, through one or more of the plurality of openings, into the second fluid chamber, and through the housing outlet;
a sensing device configured to generate one or more signals indicative of a level of urine in the second fluid chamber; and
a processor configured to receive said one or more signals and determine a urine inflow rate based on said one or more signals.

15. The system of claim 14, wherein the sensing device comprises one or more electrodes arranged within the second fluid chamber.

16. The system of claim 14, wherein:
the sensing device comprises a circuit board arranged within the second fluid chamber, said circuit board being a first circuit board of the system;
the system further comprises a second circuit board and a cable electrically connecting the second circuit board to the first circuit board, the second circuit board comprising said processor; and
the first circuit board is configured to provide said one or more signals to the processor via said cable.

17. The system of claim 16, wherein the first circuit board comprises a plurality of electrodes spaced from one another along a height of the first circuit board, and wherein said processor is configured to determine which of the plurality of electrodes are immersed in urine.

18. The system of claim 14, wherein the bottom wall of the housing is sloped toward said housing outlet.

19. The system of claim 14, wherein the housing further comprises a plurality of discharge holes along the outer wall, wherein at least some of the plurality of discharge holes are aligned with another and are arranged along a line that extends through a portion of the housing outlet.

20. The system of claim 14, wherein the inner wall is at least partially cylindrical.

21. The system of claim 14, wherein the inner wall comprises a first end arranged proximate the top end of the housing and a bottom end arranged at the bottom wall of the housing, and wherein the plurality of openings are arranged closer to the first end than the second end.

22. The system of claim 14, further comprising a first fluid chamber surface surrounded by the inner wall and partially defining said first fluid chamber, wherein the system is configured such that urine flows onto said first fluid chamber surface before flowing through said plurality of openings.

23. A system for determining urine inflow rate, the system comprising:
a bowl configured for placement on a portion of a toilet, the bowl comprising a bowl outlet and an interior bowl surface configured to guide urine that flows into the bowl toward the bowl outlet;
a housing configured to receive urine from the bowl, the housing comprising a top end configured to be attached to the bowl proximate the bowl outlet, a bottom end opposite the top end, an outer wall, a bottom wall, a fluid chamber at least partially defined by the outer wall and the bottom wall, and a housing outlet arranged proximate the bottom end;
a dome-shaped part arranged proximate the top end of the housing and the bowl outlet when the housing is attached to the bowl, the dome-shaped part configured to provide a contact surface for urine, the system configured such that urine that contacts said interior bowl surface or the dome-shaped part is guided towards the bowl outlet, into the fluid chamber, and through the housing outlet;
a sensing device configured to generate one or more signals indicative of a level of urine in the fluid chamber; and
a processor configured to receive said one or more signals and determine a urine inflow rate based on said one or more signals.

24. The system of claim 23, wherein:
the housing further comprises an inner wall arranged within and spaced from the outer wall;
the bottom wall extends between the outer and inner walls; and
the fluid chamber is at least partially defined between the outer and inner walls and above the bottom wall.

25. The system of claim 24, wherein:
the inner wall comprises at least one opening; and
the system is further configured such that urine flows through the at least one opening of the inner wall before entering the fluid chamber.

26. The system of claim 23, wherein:
the sensing device comprises a circuit board arranged within the fluid chamber, said circuit board being a first circuit board of the system;
the system further comprises a second circuit board and a cable electrically connecting the second circuit board to the first circuit board, the second circuit board comprising said processor; and
the first circuit board is configured to provide said one or more signals to the processor via said cable.

27. The system of claim 26, wherein the first circuit board comprises a plurality of electrodes spaced from one another along a height of the first circuit board, and wherein said processor is configured to determine which of the plurality of electrodes are immersed in urine.

28. A system for determining liquid inflow rate, the system comprising:
a housing configured to receive liquid from a bowl that is configured for placement on a toilet, the housing comprising a top end configured to be attached to the bowl, a bottom end opposite the top end, an outer wall, an inner wall arranged within and spaced from the outer wall, a first fluid chamber at least partially defined by the inner wall and proximate to the top end of the housing, at least one opening arranged along the inner wall, a bottom wall extending between the outer and inner walls, a second fluid chamber defined at least partially between the outer and inner walls and above the bottom wall, and a housing outlet proximate the bottom end, the housing configured such that liquid received from the bowl is guided into the first fluid chamber, through the at least one opening in the inner wall, into the second fluid chamber, and through the housing outlet;
a sensing device configured to generate one or more signals indicative of a level of liquid in the second fluid chamber; and
a processor configured to receive said one or more signals and determine a liquid inflow rate based on said one or more signals.

29. The system of claim 28, further comprising the bowl, wherein the bowl is configured for placement on a portion of a toilet and comprises a bowl outlet and an interior bowl surface configured to guide liquid that flows into the bowl toward the bowl outlet, wherein the top end of the housing is configured to be attached to the bowl proximate the bowl outlet.

30. The system of claim 29, further comprising a dome-shaped part arranged above the first fluid chamber and proximate the bowl outlet when the top end of the housing is attached to the bowl, the dome-shaped part configured to provide a contact surface for liquid, wherein the system is further configured such that liquid that contacts said interior bowl surface or the dome-shaped part is guided towards the bowl outlet and into the first fluid chamber.

31. The system of claim 28, wherein:
the sensing device comprises a circuit board arranged within the second fluid chamber, said circuit board being a first circuit board of the system;
the system further comprises a second circuit board and a cable electrically connecting the second circuit board to the first circuit board, the second circuit board comprising said processor; and
the first circuit board is configured to provide said one or more signals to the processor via said cable.

32. The system of claim 28, wherein the inner wall comprises a first end arranged proximate the top end of the housing and a bottom end arranged at the bottom wall of the housing, and wherein the at least one opening in the inner wall is arranged closer to the first end than the second end.

33. The system of claim 28, wherein said at least one opening comprises a plurality of openings arranged along the inner wall.
